(19) United States
(12) Patent Application Publication
(10) Pub. No.: US 2011/0137081 A1
(43) Pub. Date: Jun. 9, 2011

(54) Continuous Method For Producing Amides Of Low Aliphatic Carboxylic Acids

(75) Inventors: Matthias Krull, Harxheim (DE); Roman Morschhaeuser, Mainz (DE); Michael Seebach, Hofheim (DE); Hans Juergen Scholz, Alzenau (DE)

(73) Assignee: CLARIANT FINANCE (BVI) LIMITED, Tortola (VG)

(21) Appl. No.: 12/935,683

(22) PCT Filed: Mar. 18, 2009
(86) PCT No.: PCT/EP2009/001990

§ 371 Date: Dec. 8, 2010

(30) Foreign Application Priority Data
Apr. 4, 2008 (DE) 102008017218.9

(51) Int. Cl. C07C 231/02; C07C 231/08

(52) U.S. Cl. 564/139; 564/133; 564/138

(57) ABSTRACT

The invention relates to a continuous method for producing amides, according to which at least one carboxylic acid of formula (I)

R³—COON (I)

wherein R³ is hydrogen or an optionally substituted alkyl group comprising between 1 and 4 carbon atoms, is reacted with at least one amine of formula (II)

HNR¹R² (II)

wherein R¹ and R² are independently hydrogen or a hydrocarbon group comprising between 1 and 100 C atoms, to form an ammonium salt, and said ammonium salt is then reacted to form a carboxylic acid amide, under microwave irradiation in a reaction pipe, the longitudinal axis of the pipe being oriented in the direction of propagation of the microwaves of a monomode microwave applicator.

Amides of lower aliphatic carboxylic acids are of very great interest as chemical raw materials. For instance, various amides find use as intermediates for the production of pharmaceuticals and agrochemicals. The tertiary amides in particular are aprotic polar liquids with outstanding dissolving power. They are used, inter alia, to produce fibers and films, and as a reaction medium. For example, they are used as solvents for polyacrylonitrile and other polymers, as a stripping compound, extractant, catalyst and as a crystallization aid.

The industrial preparation typically involves reacting a reactive derivative of a carboxylic acid, such as acid anhydride, acid chloride or ester, with an amine. This leads firstly to high production costs and secondly to undesired accompanying products, for example salts or acids which have to be removed and disposed of or worked up. For example, the Schotten-Baumann synthesis, by which numerous carboximides are prepared on the industrial scale, forms equimolar amounts of sodium chloride. The desirable direct thermal condensation of acid and amine requires very high temperatures and long reaction times, but only moderate yields are obtained (J. Am. Chem. Soc., 59 (1937), 401-402). Moreover, the separation of acid used and amide formed is often extremely complex since the two frequently have very similar boiling points and additionally form azeotropes.

GB-414 366 discloses a process for preparing substituted amides by thermal condensation. In the examples, relatively high-boiling carboxylic acids are reacted with gaseous secondary amines at temperatures of 200-250° C. The crude products are purified by means of distillation or bleaching.

GB-719 792 discloses a process for preparing dimethylacylamides, in which a C₂-C₄-carboxylic acid and dimethylamine are converted in excess dimethylacyl-amide, such that the content of acid in the reaction mixture remains below the concentration of the azeotrope of acid and dimethylacylamide.

Particular problems with these preparation processes are very long reaction times to achieve a conversion of commercial interest and the corrosiveness of the reaction mixtures composed of acid, amine, amide and water of reaction, which severely attack or dissolve metallic reaction vessels at the high reaction temperatures required. The metal contents introduced into the products as a result are very undesired since they impair the product properties not only with regard to the color thereof, but also catalyze decomposition reactions and hence reduce the yield. The latter problem can be partly avoided by means of specific reaction vessels made of highly corrosion-resistant materials, or with appropriate coatings, which, however, requires long reaction times and hence leads to products of impaired color. Examples of undesired side reactions include oxidation of the amine, thermal disproportionation of secondary amines to primary and tertiary amine, and decarboxylation of the carboxylic acid. All these side reactions lower the yield of target product.

A more recent approach to the synthesis of amides is the microwave-supported conversion of carboxylic acids and amines to amides.

Vázquez-Tato, Synlett 1993, 506, discloses the use of microwaves as a heat source for the preparation of amides from carboxylic acids and arylaliphatic amines via the ammonium salts. The syntheses were effected on the mmol scale.

Gelens et al., Tetrahedron Letters 2005, 46(21), 3751-3754, discloses a multitude of amides which have been synthesized with the aid of microwave radiation. The syntheses were effected in 10 ml vessels.

Goretzki et al., Macromol. Rapid Commun. 2004, 25, 513-516, discloses the microwave-supported synthesis of various (meth)acrylamides directly from (meth)acrylic acid and primary amines.

The scaleup of such microwave-supported reactions from the laboratory to an industrial scale and hence the development of plants suitable for production of several tonnes, for example several tens, several hundreds or several thousands of tonnes, per year with space-time yields of interest for industrial scale applications has, however, not been achieved to date. One reason for this is the penetration depth of microwaves into the reaction mixture, which is typically limited to several millimeters to a few centimeters, and causes restriction to small vessels especially in reactions performed in batchwise processes, or leads to very long reaction times in stirred reactors. The occurrence of discharge processes and plasma formation places tight limits on an increase in the field strength, which is desirable for the irradiation of large amounts of substance with microwaves, especially in the multimode units used with preference to date for scaleup of chemical reactions. Moreover, the inhomogeneity of the microwave field, which leads to local overheating of the reaction mixture and is caused by more or less uncontrolled reflections of the microwaves injected into the microwave oven at the walls thereof and the reaction mixture, presents problems in the scaleup in the multimode microwave units typically used. In addition, the microwave absorption coefficient of the reaction mixture, which often changes during the reaction, presents difficulties with regard to a safe and reproducible reaction regime.

Chen et al., J. Chem. Soc., Chem. Commun., 1990, 807-809, describe a continuous laboratory microwave reactor, in which the reaction mixture is conducted through a Teflon pipe coil mounted in a microwave oven. A similar continuous laboratory microwave reactor is described by Cablewski et al., J. Org. Chem. 1994, 59, 3408-3412 for performance of a wide variety of different chemical reactions. In neither case, however, does the multimode microwave allow upscaling to the industrial scale range. The efficacy thereof with regard to the microwave absorption of the reaction mixture is low owing to the microwave energy being more or less homogeneously distributed over the applicator space in multimode microwave applicators and not focused on the pipe coil. A significant increase in the microwave power injected leads to undesired plasma discharges. In addition, the spatial inhomogeneities in the microwave field which change with time and are referred to as hotspots make a safe and reproducible reaction regime on a large scale impossible.

Additionally known are monomode or single-mode microwave applicators, in which a single wave mode is employed, which propagates in only one three-dimensional direction and is focused onto the reaction vessel by waveguides of exact dimensions. These instruments do allow high local field strengths, but, owing to the geometric requirements (for example, the intensity of the electrical field is at its greatest at the wave crests thereof and approaches zero at the nodes), have to date been restricted to small reaction volumes (≦50 ml) on the laboratory scale.

A process was therefore sought for preparing amides of lower carboxylic acids, in which carboxylic acid and amine can also be converted on the industrial scale under microwave irradiation to the amide. At the same time, maximum, i.e. up to quantitative, conversion rates shall be achieved. The process shall additionally enable a very energy-saving preparation of the carboxamides, which means that the microwave power used shall be absorbed substantially quantitatively by the reaction mixture and the process shall thus give a high energetic efficiency. At the same time, only minor amounts of by-products, if any, shall be obtained. The amides shall also have a minimum metal content and a low intrinsic color. In addition, the process shall ensure a safe and reproducible reaction regime.

It has been found that, surprisingly, amides of lower carboxylic acids can be prepared in industrially relevant amounts by direct reaction of carboxylic acids with amines in a continuous process by only briefly heating by means of irradiation with microwaves in a reaction tube whose longitudinal axis is in the direction of propagation of the microwaves of a monomode microwave applicator. At the same time, the microwave energy injected into the microwave applicator is virtually quantitatively absorbed by the reaction mixture. The process according to the invention additionally has a high level of safety in the performance and offers high reproducibility of the reaction conditions established. The amides prepared by the process according to the invention exhibit a high purity and low intrinsic color not obtainable in comparison to by conventional preparation processes without additional process steps.

The invention provides a continuous process for preparing amides by reacting at least one carboxylic acid of the formula I

R³—COON (I)

in which R³ is hydrogen or an optionally substituted alkyl group having 1 to 4 carbon atoms

with at least one amine of the formula II

HNR¹R² (II)

in which R¹ and R² are each independently hydrogen or a hydrocarbon radical having 1 to 100 carbon atoms

to give an ammonium salt and then converting this ammonium salt to the carboxamide under microwave irradiation in a reaction tube whose longitudinal axis is in the direction of propagation of the microwaves from a monomode microwave applicator.

The invention further provides carboxamides with low metal content, prepared by reaction of at least one carboxylic acid of the formula I

R³—COOH (I)

in which R³ is hydrogen or an optionally substituted alkyl group having 1 to 4 carbon atoms,

with at least one amine of the formula

HNR¹R² (II)

in which R¹ and R² are each independently hydrogen or a hydrocarbon radical having 1 to 100 carbon atoms,

to give an ammonium salt and then converting this ammonium salt to the carboxamide under microwave irradiation in a reaction tube longitudinal axis whose is in the direction of propagation of the microwaves from a monomode microwave applicator.

R³ is preferably a saturated alkyl radical having 1, 2, 3 or 4 carbon atoms. It may be linear or else branched. The carboxyl group may be bonded to a primary, secondary or, as in the case of pivalic acid, tertiary carbon atom. In a preferred embodiment, the alkyl radical is an unsubstituted alkyl radical. In a further preferred embodiment, the alkyl radical bears one to nine, preferably one to five, for example two, three or four, further substituents. Such substituents may be, for example, C₁-C₅-alkoxy, for example methoxy, ester, amide, carboxyl, cyano, nitrile, nitro and/or C₅-C₂₀-aryl groups, for example phenyl groups, with the proviso that the substituents are stable under the reaction conditions and do not enter into any side reactions, for example elimination reactions. The C₅-C₂₀ aryl groups may themselves in turn bear substituents. Such substituents may, for example, be C₁-C₂₀-alkyl, C₂-C₂₀-alkenyl, C₁-C₅-alkoxy, for example methoxy, ester, amide, carboxyl, cyano, nitrile and/or nitro groups. However, the alkyl radical bears at most as many substituents as it has valences. In a specific embodiment, the alkyl radical R³ bears further carboxyl groups. Thus, the process according to the invention is equally suitable for reacting carboxylic acids having, for example, two or more carboxyl groups. The reaction of such polycarboxylic acids with primary amines by the process according to the invention can also form imides. Suitable aliphatic carboxylic acids are, for example, formic acid, acetic acid, propionic acid, butyric acid, isobutyric acid, pentanoic acid, isopentanoic acid, pivalic acid, succinic acid, butanetetracarboxylic acid, phenylacetic acid, (2-bromophenyl)acetic acid, (methoxyphenyl)acetic acid, (dimethoxyphenyl)acetic acid, 2-phenylpropionic acid, 3-phenylpropionic acid, 3-(4-hydroxyphenyl)propionic acid, 4-hydroxy-phenoxyacetic acid and mixtures thereof. Carboxylic acids particularly preferred in accordance with the invention are formic acid, acetic acid and propionic acid, and also phenylacetic acid and the derivatives thereof substituted on the aryl radical.

The process according to the invention is preferentially suitable for preparation of secondary amides, i.e. for reaction of carboxylic acids with amines in which R¹ is a hydrocarbon radical having 1 to 100 carbon atoms and R² is hydrogen.

The process according to the invention is more preferentially suitable for preparation of tertiary amides, i.e. for reaction of carboxylic acids with amines in which both R¹ and R² radicals are independently a hydrocarbon radical having 1 to 100 carbon atoms. The R¹ and R² radicals may be the same or different. In a particularly preferred embodiment, R¹ and R² are the same.

In a first preferred embodiment, R¹ and/or R² are each independently an aliphatic radical. It has preferably 1 to 24, more preferably 2 to 18 and especially 3 to 6 carbon atoms. The aliphatic radical may be linear, branched or cyclic. It may additionally be saturated or unsaturated. The hydrocarbon radical may bear substituents. Such substituents may, for example, be hydroxyl, C₁-C₅-alkoxy, alkoxyalkyl, cyano, nitrile, nitro and/or C₅-C₂₀-aryl groups, for example phenyl radicals. The C₅-C₂₀-aryl groups may in turn optionally be substituted by halogen atoms, C₁-C₂₀-alkyl, C₂-C₂₀-alkenyl, hydroxyl, C₁-C₅-alkoxy, for example methoxy, ester, amide, cyano, nitrile and/or nitro groups. Particularly preferred aliphatic radicals are methyl, ethyl, hydroxyethyl, n-propyl, isopropyl, hydroxypropyl, n-butyl, isobutyl and tert-butyl, hydroxybutyl, n-hexyl, cyclohexyl, n-octyl, n-decyl, n-dodecyl, tridecyl, isotridecyl, tetradecyl, hexadecyl, octadecyl and methylphenyl. In a particularly preferred embodiment, R¹ and/or R² are each independently hydrogen, a C₁-C₆-alkyl, C₂-C₆-alkenyl or C₃-C₆-cycloalkyl radical, and especially an alkyl radical having 1, 2 or 3 carbon atoms. These radicals may bear up to three substituents.

In a further preferred embodiment, R¹ and R² together with the nitrogen atom to which they are bonded form a ring. This ring has preferably 4 or more, for example 4, 5, 6 or more, ring members. Preferred further ring members are carbon, nitrogen, oxygen and sulfur atoms. The rings may themselves in turn bear substituents, for example alkyl radicals. Suitable ring structures are, for example, morpholinyl, pyrrolidinyl, piperidinyl, imidazolyl and azepanyl radicals.

In a further preferred embodiment, R¹ and/or R² are each independently an optionally substituted C₆-C₁₂ aryl group or an optionally substituted heteroaromatic group having 5 to 12 ring members.

In a further preferred embodiment, R¹ and/or R² are each independently an alkyl radical interrupted by a heteroatom. Particularly preferred heteroatoms are oxygen and nitrogen.

For instance, R¹ and R² are preferably each independently radicals of the formula III

—(R⁴—O)ₙ—R⁵ (III)

in which

R⁴ is an alkylene group having 2 to 6 carbon atoms, and preferably having 2 to 4 carbon atoms, for example ethylene, propylene, butylene or mixtures thereof,

R⁵ is hydrogen, a hydrocarbon radical having 1 to 24 carbon atoms or a group of the formula —NR¹⁰R¹¹,

n is an integer from 2 to 50, preferably from 3 to 25 and especially from 4 to 10, and

R¹⁰, R¹¹ are each independently hydrogen, an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the poly(oxyalkylene) units derive from alkylene oxide units having 2 to 6 carbon atoms or R¹⁰ and R¹¹ together with the nitrogen atom to which they are bonded form a ring having 4, 5, 6 or more ring members.

Additionally preferably, R¹ and/or R² are each independently radicals of the formula IV

—[R⁶—N(R⁷)]ₘ—(R⁷) (IV)

in which

R⁶ is an alkylene group having 2 to 6 carbon atoms and preferably having 2 to 4 carbon atoms, for example ethylene, propylene or mixtures thereof,

each R⁷ is independently hydrogen, an alkyl or hydroxyalkyl radical having up to 24 carbon atoms, for example 2 to 20 carbon atoms, a polyoxyalkylene radical —(R⁴—O)ₚ—R⁵, or a polyiminoalkylene radical —[R⁶—N(R⁷)]_{q}—(R⁷), where R⁴, R⁵, R⁶ and R⁷ are each as defined above and q and p are each independently 1 to 50, and

m is from 1 to 20 and preferably 2 to 10, for example three, four, five or six. The radicals of the formula IV preferably contain 1 to 50 and especially 2 to 20 nitrogen atoms.

According to the stoichiometric ratio between carboxylic acid (I) and polyamine (IV), one or more amino groups which each bear at least one hydrogen atom are converted to the carboxamide. In the reaction of polycarboxylic acids with polyamines of the formula IV, the primary amino groups in particular can also be converted to imides.

For the inventive preparation of primary amides, instead of ammonia, preference is given to using nitrogen compounds which eliminate ammonia gas when heated. Examples of such nitrogen compounds are urea and formamide.

Examples of suitable amines are ammonia, methylamine, ethylamine, ethanolamine, propylamine, propanolamine, butylamine, hexylamine, cyclohexylamine, octylamine, decylamine, dodecylamine, tetradecylamine, hexadecylamine, octadecylamine, dimethylamine, diethylamine, diethanolamine, ethylmethylamine, di-n-propylamine, diisopropylamine, dicyclohexylamine, didecylamine, didodecylamine, ditetradecylamine, dihexadecylamine, dioctadecylamine, benzylamine, phenylethylamine, ethylenediamine, diethylenetriamine, triethylenetetramine, tetraethylenepentamine and mixtures thereof. Among these, particular preference is given to dimethylamine, diethylamine, di-n-propylamine, diisopropylamine and ethylmethylamine.

The process is especially suitable for preparing N,N-dimethylformamide, N,N-dimethylacetamide, N,N-dimethylpropionamide, N,N-dimethylbutyramide, N,N-diethylformamide, N,N-diethylacetamide, N,N-diethylpropionamide, N,N-diethylbutyramide, N,N-dipropylacetamide, N,N-dimethyl(phenyl)acetamide, N,N-dimethyl(p-methoxyphenyl)acetamide and N,N-dimethyl-2-phenylpropionic acid.

In the process according to the invention, aliphatic carboxylic acid and amine can be reacted with one another in any desired ratios. The reaction between carboxylic acid and amine is preferably effected with molar ratios of 10:1 to 1:100, preferably of 2:1 to 1:10, especially of 1.2:1 to 1:3, based in each case on the molar equivalents of carboxyl groups. In a specific embodiment, carboxylic acid and amine are used in equimolar amounts.

In many cases, it has been found to be advantageous to work with an excess of amine, i.e. molar ratios of amine to carboxyl groups of at least 1.01:1.00 and especially between 50:1 and 1.02:1, for example between 10:1 and 1.1:1. This converts the carboxyl groups virtually quantitatively to the amide. This process is particularly advantageous when the amine used is volatile. “Volatile” means here that the amine has a boiling point at standard pressure of preferably below 200° C., for example below 160° C., and can thus be removed by distillation from the amide.

In the case that R¹ and/or R² is a hydrocarbon radical substituted by one or more hydroxyl groups, the reaction between carboxylic acid and amine is effected with molar ratios of 1:1 to 1:100, preferably of 1:1.001 to 1:10 and especially of 1:1.01 to 1:5, for example of 1:1.1 to 1:2, based in each case on the molar equivalents of carboxyl groups and amino groups in the reaction mixture.

The inventive preparation of the amides proceeds by reaction of carboxylic acid and amine to give the ammonium salt and subsequent irradiation of the salt with microwaves in a reaction tube whose longitudinal axis is in the direction of propagation of the microwaves in a monomode microwave applicator.

The salt is preferably irradiated with microwaves in a substantially microwave-transparent reaction tube within a hollow conductor connected to a microwave generator. The reaction tube is preferably aligned axially with the central axis of symmetry of the hollow conductor.

The hollow conductor which functions as the microwave applicator is preferably configured as a cavity resonator. Additionally preferably, the microwaves unabsorbed in the hollow conductor are reflected at the end thereof. Configuration of the microwave applicator as a resonator of the reflection type achieves a local increase in the electrical field strength at the same power supplied by the generator and increased energy exploitation.

The cavity resonator is preferably operated in E₀₁ₙ mode where n is an integer and specifies the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value 0 toward the outer surface. This field configuration is rotationally symmetric about the central axis of symmetry. According to the desired flow rate of the reaction mixture through the reaction tube, the temperature required and the residence time required in the resonator, the length of the resonator is selected relative to the wavelength of the microwave radiation used. n is preferably an integer from 1 to 200, more preferably from 2 to 100, particularly from 4 to 50 and especially from 3 to 20, for example 3, 4, 5, 6, 7 or 8.

The microwave energy can be injected into the hollow conductor which functions as the microwave applicator through holes or slots of suitable dimensions. In an embodiment particularly preferred in accordance with the invention, the ammonium salt is irradiated with microwaves in a reaction tube present in a hollow conductor with a coaxial transition of the microwaves. Microwave devices particularly preferred from this process are formed from a cavity resonator, a coupling device for injecting a microwave field into the cavity resonator and with one orifice each on two opposite end walls for passage of the reaction tube through the resonator. The microwaves are preferably injected into the cavity resonator by means of a coupling pin which projects into the cavity resonator. The coupling pin is preferably configured as a preferably metallic inner conductor tube which functions as a coupling antenna. In a particularly preferred embodiment, this coupling pin projects through one of the end orifices into the cavity resonator. The reaction tube more preferably adjoins the inner conductor tube of the coaxial transition, and is especially conducted through the cavity thereof into the cavity resonator. The reaction tube is preferably aligned axially with a central axis of symmetry of the cavity resonator, for which the cavity resonator preferably has one central orifice each on two opposite end walls for passage of the reaction tube.

The microwaves can be fed into the coupling pin or into the inner conductor tube which functions as a coupling antenna, for example, by means of a coaxial connecting line. In a preferred embodiment, the microwave field is supplied to the resonator via a hollow conductor, in which case the end of the coupling pin projecting out of the cavity resonator is conducted into the hollow conductor through an orifice in the wall of the hollow conductor, and takes microwave energy from the hollow conductor and injects it into the resonator.

In a specific embodiment, the salt is irradiated with microwaves in a microwave-transparent reaction tube which is axially symmetric within an E₀₁ₙ round hollow conductor with a coaxial transition of the microwaves. In this case, the reaction tube is conducted through the cavity of an inner conductor tube which functions as a coupling antenna into the cavity resonator. In a further preferred embodiment, the salt is irradiated with microwaves in a microwave-transparent reaction tube which is conducted through an E₀₁ₙ cavity resonator with axial feeding of the microwaves, the length of the cavity resonator being such that n=2 or more field maxima of the microwave form. In a further preferred embodiment, the salt is irradiated with microwaves in a microwave-transparent reaction tube which is axially symmetric within a circular cylindrical E₀₁ₙ cavity resonator with a coaxial transition of the microwaves, the length of the cavity resonator being such that n=2 or more field maxima of the microwave form.

Microwave generators, for example the magnetron, the klystron and the gyrotron, are known to those skilled in the art.

The reaction tubes used to perform the process according to the invention are preferably manufactured from substantially microwave-transparent, high-melting material. Particular preference is given to using nonmetallic reaction tubes. “Substantially microwave-transparent” is understood here to mean materials which absorb a minimum amount of microwave energy and convert it to heat. A measure employed for the ability of a substance to absorb microwave energy and convert it to heat is often the dielectric loss factor tan δ=ε″/ε′. The dielectric loss factor tan δ is defined as the ratio of dielectric loss ε″ to dielectric constant ε′. Examples of tan δ values of different materials are reproduced, for example, in D. Bogdal, Microwave-assisted Organic Synthesis, Elsevier 2005. For reaction tubes suitable in accordance with the invention, materials with tan δ values measured at 2.45 GHz and 25° C. of less than 0.01, particularly less than 0.005 and especially less than 0.001 are preferred. Preferred microwave-transparent and thermally stable materials include primarily mineral-based materials, for example quartz, aluminum oxide, zirconium oxide and the like. Other suitable tube materials are thermally stable plastics, such as especially fluoropolymers, for example Teflon, and industrial plastics such as polypropylene, or polyaryl ether ketones, for example glass fiber-reinforced polyetheretherketone (PEEK). In order to withstand the temperature conditions during the reaction, minerals, such as quartz or aluminum oxide, coated with these plastics have been found to be especially suitable as reactor materials.

Reaction tubes particularly suitable for the process according to the invention have an internal diameter of 1 mm to approx. 50 cm, especially between 2 mm and 35 cm for example between 5 mm and 15 cm. Reaction tubes are understood here to mean vessels whose ratio of length to diameter is greater than 5, preferably between 10 and 100 000, more preferably between 20 and 10 000, for example between 30 and 1000. A length of the reaction tube is understood here to mean the length of the reaction tube over which the microwave irradiation proceeds. Baffles and/or other mixing elements can be incorporated into the reaction tube.

E₀₁ cavity resonators particularly suitable for the process according to the invention preferably have a diameter which corresponds to at least half the wavelength of the microwave radiation used. The diameter of the cavity resonator is preferably 1.0 to 10 times, more preferably 1.1 to 5 times and especially 2.1 to 2.6 times half the wavelength of the microwave radiation used. The E₀₁ cavity resonator preferably has a round cross section, which is also referred to as an E₀₁ round hollow conductor. It more preferably has a cylindrical shape and especially a circular cylindrical shape.

The reaction tube is typically provided at the inlet with a metering pump and a manometer, and at the outlet with a pressure-retaining device and a heat exchanger. This makes possible reactions within a very wide pressure and temperature range.

The conversion of amine and carboxylic acid to the ammonium salt can be performed continuously, batchwise or else in semibatchwise processes. Thus, the preparation of the ammonium salt can be performed in an upstream (semi)-batchwise process, for example in a stirred vessel. The ammonium salt is preferably obtained in situ and not isolated. In a preferred embodiment, the amine and carboxylic acid reactants, each independently optionally diluted with solvent, are only mixed shortly before entry into the reaction tube. For instance, it has been found to be particularly useful to undertake the reaction of amine and carboxylic acid to give the ammonium salt in a mixing zone, from which the ammonium salt, optionally after intermediate cooling, is conveyed into the reaction tube. Additionally preferably, the reactants are supplied to the process according to the invention in liquid form. For this purpose, it is possible to use relatively high-melting and/or relatively high-viscosity reactants, for example in the molten state and/or admixed with solvent, for example in the form of a solution, dispersion or emulsion. A catalyst can, if used, be added to one of the reactants or else to the reactant mixture before entry into the reaction tube. It is also possible to convert solid, pulverulent and heterogeneous systems by the process according to the invention, in which case merely appropriate industrial apparatus for conveying the reaction mixture is required.

The ammonium salt can be fed into the reaction tube either at the end conducted through the inner conductor tube or at the opposite end.

By variation of tube cross section, length of the irradiation zone (this is understood to mean the length of the reaction tube in which the reaction mixture is exposed to microwave radiation), flow rate, geometry of the cavity resonator, the microwave power injected and the temperature achieved, the reaction conditions are established such that the maximum reaction temperature is attained as rapidly as possible and the residence time at maximum temperature remains sufficiently short that as low as possible a level of side reactions or further reactions occurs. To complete the reaction, the reaction mixture can pass through the reaction tube more than once, optionally after intermediate cooling. In many cases, it has been found to be useful when the reaction product is cooled immediately after leaving the reaction tube, for example by jacket cooling or decompression. In the case of slower reactions, it has often been found to be useful to keep the reaction product at reaction temperature for a certain time after it leaves the reaction tube.

The advantages of the process according to the invention lie in very homogeneous irradiation of the reaction mixture in the center of a symmetric microwave field within a reaction tube, the longitudinal axis of which is in the direction of propagation of the microwaves of a monomode microwave applicator and especially within an E₀₁ cavity resonator, for example with a coaxial transition. The inventive reactor design allows the performance of reactions also at very high pressures and/or temperatures. By increasing the temperature and/or pressure, a significant rise in the degree of conversion and yield is observed even compared to known microwave reactors, without this resulting in undesired side reactions and/or discoloration. Surprisingly, this achieves a very high efficiency in the exploitation of the microwave energy injected into the cavity resonator, which is typically more than 50%, often more than 80%, in some cases more than 90% and in special cases more than 95%, for example more than 98%, of the microwave power injected, and therefore gives economic and also ecological advantages over conventional preparation processes, and also over prior art microwave processes.

The process according to the invention additionally allows a controlled, safe and reproducible reaction regime. Since the reaction mixture in the reaction tube is moved parallel to the direction of propagation of the microwaves, known overheating phenomena as a result of uncontrolled field distributions, which lead to local overheating as a result of changing intensities of the field, for example in wave crests and nodes, are balanced out by the flowing motion of the reaction mixture. The advantages mentioned also allow working with high microwave powers of, for example, more than 10 kW or more than 100 kW and thus, in combination with only a short residence time in the cavity resonator, accomplishment of large production amounts of 100 or more tonnes per year in one plant.

It was particularly surprising that, in spite of the only very short residence time of the ammonium salt in the microwave field in the flow tube with continuous flow, very substantial amidation takes place with conversions generally of more than 80%, often even more than 90%, for example more than 95%, based on the component used in deficiency, without significant formation of by-products. In the case of a corresponding conversion of these ammonium salts in a flow tube, of the same dimensions with thermal jacket heating, achievement of suitable reaction temperatures requires extremely high wall temperatures which lead to formation of colored species, but only minor amide formation in the same time interval. In addition, the products prepared by the process according to the invention have very low metal contents, without requiring a further workup of the crude products. For instance, the metal contents of the products prepared by the process according to the invention, based on iron as the main element, are typically less than 25 ppm, preferably less than 15 ppm, especially less than 10 ppm, for example between 0.01 and 5 ppm, of iron.

The temperature rise caused by the microwave radiation is preferably limited to a maximum of 500° C., for example, by regulating the microwave intensity of the flow rate and/or by cooling the reaction tube, for example by means of a nitrogen stream. It has been found to be particularly useful to perform the reaction at temperatures between 150 and a maximum of 400° C. and especially between 180 and a maximum of 300° C., for example at temperatures between 200 and 270° C.

The duration of the microwave irradiation depends on various factors, for example the geometry of the reaction tube, the microwave energy injected, the specific reaction and the desired degree of conversion. Typically, the microwave irradiation is undertaken over a period of less than 30 minutes, preferably between 0.01 second and 15 minutes, more preferably between 0.1 second and 10 minutes and especially between 1 second and 5 minutes, for example between 5 seconds and 2 minutes. The intensity (power) of the microwave radiation is adjusted such that the reaction mixture has the desired maximum temperature when it leaves the cavity resonator. In a preferred embodiment, the reaction product, directly after the microwave irradiation has ended, is cooled as rapidly as possible to temperatures below 120° C., preferably below 100° C. and especially below 60° C.

The reaction is preferably performed at pressures between 0.01 and 500 bar and more preferably between 1 bar (atmospheric pressure) and 150 bar and especially between 1.5 bar and 100 bar, for example between 3 bar and 50 bar. It has been found to be particularly useful to work under elevated pressure, which involves working above the boiling point (at standard pressure) of the reactants or products, or of any solvent present, and/or above the water of reaction formed during the reaction. The pressure is more preferably adjusted to a sufficiently high level that the reaction mixture remains in the liquid state during the microwave irradiation and does not boil.

To avoid side reactions and to prepare products of maximum purity, it has been found to be useful to handle reactants and products in the presence of an inert protective gas, for example nitrogen, argon or helium.

In a preferred embodiment, the reaction is accelerated or completed by working in the presence of dehydrating catalysts. Preference is given to working in the presence of an acidic inorganic, organometallic or organic catalyst, or mixtures of two or more of these catalysts.

Acidic inorganic catalysts in the context of the present invention include, for example, sulfuric acid, phosphoric acid, phosphonic acid, hypophosphorous acid, aluminum sulfide hydrate, alum, acidic silica gel and acidic aluminum hydroxide. In addition, for example, aluminum compounds of the general formula Al(OR¹⁵)₃ and titanates of the general formula Ti(OR¹⁵)₄ are usable as acidic inorganic catalysts, where R¹⁵ radicals may each be the same or different and are each independently selected from C₁-C₁₀ alkyl radicals, for example methyl, ethyl, n-propyl, isopropyl, n-butyl, isobutyl, sec-butyl, tert-butyl, n-pentyl, isopentyl, sec-pentyl, neo-pentyl, 1,2-dimethylpropyl, isoamyl, n-hexyl, sec-hexyl, n-heptyl, n-octyl, 2-ethylhexyl, n-nonyl or n-decyl, C₃-C₁₂ cycloalkyl radicals, for example cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl. The R¹⁵ radicals in Al(OR¹⁵)₃ or Ti(OR¹⁵)₄ are preferably each the same and are selected from isopropyl, butyl and 2-ethylhexyl.

Preferred acidic organometallic catalysts are, for example, selected from dialkyltin oxides (R¹⁵)₂SnO, where R¹⁵ is as defined above. A particularly preferred representative of acidic organometallic catalysts is di-n-butyltin oxide, which is commercially available as “Oxo-tin” or as Fascat® brands.

Preferred acidic organic catalysts are acidic organic compounds with, for example, phosphate groups, sulfo groups, sulfate groups or phosphonic acid groups. Particularly preferred sulfonic acids contain at least one sulfo group and at least one saturated or unsaturated, linear, branched and/or cyclic hydrocarbon radical having 1 to 40 carbon atoms and preferably having 3 to 24 carbon atoms. Especially preferred are aromatic sulfonic acids, especially alkylaromatic monosulfonic acids having one or more C₁-C₂₈ alkyl radicals and especially those having C₃-C₂₂ alkyl radicals. Suitable examples are methanesulfonic acid, butanesulfonic acid, benzenesulfonic acid, p-toluenesulfonic acid, xylenesulfonic acid, 2-mesitylenesulfonic acid, 4-ethylbenzenesulfonic acid, isopropylbenzenesulfonic acid, 4-butylbenzenesulfonic acid, 4-octylbenzenesulfonic acid; dodecylbenzenesulfonic acid, didodecylbenzenesulfonic acid, naphthalenesulfonic acid. It is also possible to use acidic ion exchangers as acidic organic catalysts, for example sulfo-containing poly(styrene) resins crosslinked with about 2 mol % of divinylbenzene.

Particular preference for the performance of the process according to the invention is given to boric acid, phosphoric acid, polyphosphoric acid and polystyrenesulfonic acids. Especially preferred are titanates of the general formula Ti(OR¹⁵)₄, and especially titanium tetrabutoxide and titanium tetraisopropoxide.

If the use of acidic inorganic, organometallic or organic catalysts is desired, in accordance with the invention, 0.01 to 10% by weight, preferably 0.02 to 2% by weight, of catalyst is used. In a particularly preferred embodiment, no catalyst is employed.

In a further preferred embodiment, the microwave irradiation is performed in the presence of acidic solid catalysts. This involves suspending the solid catalyst in the ammonium salt optionally admixed with solvent, or advantageously passing the ammonium salt optionally admixed with solvent over a fixed bed catalyst and exposing it to microwave radiation. Suitable solid catalysts are, for example, zeolites, silica gel, montmorillonite and (partly) crosslinked polystyrenesulfonic acid, which may optionally be integrated with catalytically active metal salts. Suitable acidic ion exchangers based on polystyrenesulfonic acids, which can be used as solid phase catalysts, are obtainable, for example, from Rohm & Haas under the Amberlyst® brand name.

It has been found to be useful to work in the presence of solvents in order, for example, to lower the viscosity of the reaction medium and/or to fluidize the reaction mixture if it is heterogeneous. For this purpose, it is possible in principle to use all solvents which are inert under the reaction conditions employed and do not react with the reactants or the products formed. An important factor in the selection of suitable solvents is the polarity thereof, which firstly determines the dissolution properties and secondly the degree of interaction with microwave radiation. A particularly important factor in the selection of suitable solvents is the dielectric loss ε″ thereof. The dielectric loss ε″ describes the proportion of microwave radiation which is converted to heat in the interaction of a substance with microwave radiation. The latter value has been found to be a particularly important criterion for the suitability of a solvent for the performance of the process according to the invention. It has been found to be particularly useful to work in solvents which exhibit minimum microwave absorption and hence make only a small contribution to the heating of the reaction system. Solvents preferred for the process according to the invention have a dielectric loss ε″ measured at room temperature and 2450 MHz of less than 10 and preferably less than 1, for example less than 0.5. An overview of the dielectric loss of different solvents can be found, for example, in “Microwave Synthesis” by B. L. Hayes, CEM Publishing 2002. Suitable solvents for the process according to the invention are especially those with ε″ values less than 10, such as N-methylpyrrolidone, N,N-dimethylformamide or acetone, and especially solvents with ε″ values less than 1. Examples of particularly preferred solvents with ε″ values less than 1 are aromatic and/or aliphatic hydrocarbons, for example toluene, xylene, ethylbenzene, tetralin, hexane, cyclohexane, decane, pentadecane, decalin, and also commercial hydrocarbon mixtures, such as benzine fractions, kerosene, Solvent Naphtha, ®Shellsol AB, ®Solvesso 150, ®Solvesso 200, ®Exxsol, ®Isopar and ®Shellsol products. Solvent mixtures which have ε″ values preferably below 10 and especially below 1 are equally preferred for the performance of the process according to the invention.

In principle, the process according to the invention is also performable in solvents with higher ε″ values of, for example, 5 or higher, such as especially with ε″ values of 10 or higher. However, the accelerated heating of the reaction mixture observed requires special measures to comply with the maximum temperature.

When working in the presence of solvents, the proportion thereof in the reaction mixture is preferably between 2 and 95% by weight, especially between 5 and 90% by weight and particularly between 10 and 75% by weight, for example between 30 and 60% by weight. Particular preference is given to performing the reaction without solvents.

Microwaves refer to electromagnetic rays with a wavelength between about 1 cm and 1 m, and frequencies between about 300 MHz and 30 GHz. This frequency range is suitable in principle for the process according to the invention. For the process according to the invention, preference is given to using microwave radiation with the frequencies approved for industrial, scientific and medical applications, for example with frequencies of 915 MHz, 2.45 GHz, 5.8 GHz or 27.12 GHz.

The microwave power to be injected into the cavity resonator for the performance of the process according to the invention is especially dependent on the geometry of the reaction tube and hence of the reaction volume, and on the duration of the irradiation required. It is typically between 200 W and several hundred kW and especially between 500 W and 100 kW for example between 1 kW and 70 kW. It can be generated by means of one or more microwave generators.

In a preferred embodiment, the reaction is performed in a pressure-resistant inert tube, in which case the water of reaction which forms and possibly reactants and, if present, solvent lead to a pressure buildup. After the reaction has ended, the elevated pressure can be used by decompression for volatilization and removal of water of reaction, excess reactants and any solvent and/or to cool the reaction product. In a further embodiment, the water of reaction formed, after cooling and/or decompression, is removed by customary processes, for example phase separation, distillation, stripping, flashing and/or absorption.

To complete the conversion, it has in many cases been found to be useful to expose the crude product obtained, after removal of water of reaction and if appropriate discharge of product and/or by-product, again to microwave irradiation, in which case the ratio of the reactants used may have to be supplemented to replace consumed or deficient reactants.

Amides prepared via the inventive route are typically obtained in a purity sufficient for further use. For specific requirements, they can, however, be purified further by customary purification processes, for example distillation, recrystallization, filtration or chromatographic processes.

The process according to the invention allows a very rapid, energy-saving and inexpensive preparation of amides of lower carboxylic acids in high yields and with high purity in industrial scale amounts. The very homogeneous irradiation of the ammonium salt in the center of the rotationally symmetric microwave field allows a safe, controllable and reproducible reaction regime. At the same time, a very high efficiency in the exploitation of the incident microwave energy achieves an economic viability distinctly superior to the known preparation processes. In this process, no significant amounts of by-products are obtained. Such rapid and selective reactions cannot be achieved by conventional methods and were not to be expected solely through heating to high temperatures. The products prepared by the process according to the invention are often so pure that no further workup or further processing steps are required.

### EXAMPLES

The conversions of the ammonium salts under microwave irradiation were effected in a ceramic tube (60×1 cm) which was present in axial symmetry in a cylindrical cavity resonator (60×10 cm). On one of the end sides of the cavity resonator, the ceramic tube passed through the cavity of an inner conductor tube which functions as a coupling antenna. The microwave field with a frequency of 2.45 GHz, generated by a magnetron, was injected into the cavity resonator by means of the coupling antenna (E₀₁ cavity applicator; monomode).

The microwave power was in each case adjusted over the experiment time in such a way that the desired temperature of the reaction mixture at the end of the irradiation zone was kept constant. The microwave powers mentioned in the experiment descriptions therefore represent the mean value of the microwave power injected over time. The measurement of the temperature of the reaction mixture was undertaken directly after it had left the reaction zone (distance about 15 cm in an insulated stainless steel capillary, Ø 1 cm) by means of a Pt100 temperature sensor. Microwave energy not absorbed directly by the reaction mixture was reflected at the end side of the cavity resonator at the opposite end to the coupling antenna; the microwave energy which was also not absorbed by the reaction mixture on the return path and reflected back in the direction of the magnetron was passed with the aid of a prism system (circulator) into a water-containing vessel. The difference between energy injected and heating of this water load was used to calculate the microwave energy introduced into the reaction mixture.

By means of a high-pressure pump and of a suitable pressure-release valve, the reaction mixture in the reaction tube was placed under such a working pressure which was sufficient always to keep all reactants and products or condensation products in the liquid state. The ammonium salts prepared from carboxylic acid and amine were pumped with a constant flow rate through the reaction tube, and the residence time in the irradiation zone was adjusted by modifying the flow rate.

The products were analyzed by means of ¹H NMR spectroscopy at 500 MHz in CDCl₃. The properties were determined by means of atomic absorption spectroscopy.

### Example 1

Preparation of N,N-dimethylmethanamide (dimethylformamide)

While cooling with dry ice, 2.25 kg of dimethylamine (50 mol) from a reservoir bottle was condensed into a cold trap. Subsequently, a 10 l Büchi stirred autoclave with gas inlet tube, mechanical stirrer, internal thermometer and pressure equalizer was initially charged with 2.3 kg of formic acid (50 mol), which were cooled to 5° C. By slowly thawing the cold trap, gaseous dimethylamine was passed through the gas inlet tube into the stirred autoclave. In a strongly exothermic reaction, the formic acid N,N-dimethylammonium salt formed.

The ammonium salt thus obtained was pumped through the reaction tube continuously at 5.0 l/h at a working pressure of 35 bar and exposed to a microwave power of 1.95 kW, 93% of which was absorbed by the reaction mixture. The residence time of the reaction mixture in the irradiation zone was approx. 34 seconds. At the end of the reaction tube, the reaction mixture had a temperature of 245° C.

A conversion of 92% of theory was attained. The reaction product was virtually colorless and contained <2 ppm of iron. After distillative removal of the water of reaction, the product was isolated at a boiling temperature of 153° C. with a purity of >99.5% in 87% yield. In the bottoms remained the unreacted residues of the methanoic acid N,N-dimethylammonium salt, which were converted to the amide virtually quantitatively on renewed microwave irradiation.

### Example 2

Preparation of N,N-dimethylethanamide (dimethylacetamide)

The ammonium salt was prepared analogously to the process described in example 1. 2.4 kg (40 mol) of acetic acid and 1.9 kg (42 mol) of dimethylamine were used. The ammonium salt thus obtained was pumped through the reaction tube continuously at 4.2 l/h at a working pressure of 30-35 bar and exposed to a microwave power of 1.75 kW, 88% of which was absorbed by the reaction mixture. The residence time of the reaction mixture in the irradiation zone was approx. 40 seconds. At the end of the reaction tube, the reaction mixture had a temperature of 241° C.

Based on the acid component used, a conversion of 91% of theory was attained. The crude product was virtually colorless and contained <2 ppm of iron. Water of reaction and excess dimethylamine were removed by distillation, then the product was purified by distillation at a boiling temperature of 164-166° C. with a purity of >99% and a yield of 85%. In the bottoms remained the unreacted residues of the acetic acid N,N-dimethylammonium salt, which were converted to the amide virtually quantitatively on renewed microwave irradiation.

### Example 3

Preparation of N,N-dimethylpropanamide (dimethylpropionamide)

The ammonium salt was prepared analogously to the process described in example 1. 3.7 kg (50 mol) of propionic acid and 4.5 kg (100 mol) of dimethyl-amine were used. The ammonium salt thus obtained was pumped through the reaction tube continuously at 3.8 l/h at a working pressure of 30 bar and exposed to a microwave power of 1.90 kW, 90% of which was absorbed by the reaction mixture. The residence time of the reaction mixture in the irradiation zone was approx. 45 seconds. At the end of the reaction tube, the reaction mixture had a temperature of 260° C.

Based on the acid component used in deficiency, a conversion of 94% of theory was attained. The crude product was virtually colorless and contained <2 ppm of iron. Water of reaction and excess dimethylamine were removed by distillation.

### Example 4

Preparation of N-octylformamide

2.59 kg of octylamine (20 mol) were heated to 40° C. and admixed with 0.92 kg (20 mol) of pure formic acid. The addition of the acid was sufficiently slow that the neutralization reaction did not heat the reaction mixture above 90° C. The ammonium salt thus obtained was pumped into the reaction tube at a temperature of 90° C. In the course of this, a working pressure of 26 bar was applied, in order to prevent boiling of the components. At a delivery output of 2.8 l/h, the mixture was irradiated with a microwave power of 1.6 kW/h, 96% of which was absorbed by the reaction mixture. The average residence time of the reaction mixture in the microwave field was 61 seconds. At the end of the reaction tube, the reaction mixture had a temperature of 255° C.

Based on the acid used, a conversion of 96% was attained. No signs of corrosion were found; the iron content measured in the crude product was <2 ppm. The water of reaction was removed quantitatively by means of a thin-film evaporator.

### Example 5

Preparation of N,N-dimethyl-4-methoxyphenylacetamide

While cooling with dry ice, 2.7 kg of dimethylamine (60 mol) from a reservoir bottle were condensed into a cold trap. A 10 l Büchi stirred autoclave with gas inlet tube, mechanical stirrer, internal thermometer and pressure equalizer was initially charged with 10 kg of 4-methoxyphenylacetic acid (60 mol), which were melted at about 100° C. By slowly thawing the amine-containing cold trap, gaseous dimethylamine was introduced slowly through the gas inlet tube directly into the acid melt in the stirred autoclave. In an exothermic reaction, the 4-methoxyphenyl-acetic acid N,N-dimethylammonium salt formed. The molten ammonium salt thus obtained (95° C.) was pumped continuously through the reaction tube at 3.0 l/h at a working pressure of about 25 bar and exposed to a microwave power of 1.95 kW, 95% of which was absorbed by the reaction mixture. The residence time of the reaction mixture in the irradiation zone was approx. 57 seconds. At the end of the reaction tube, the reaction mixture had a temperature of 245° C.

Based on the acid component used, a conversion of 97% of theory was attained in the crude product. The crude product contained <2 ppm of iron and had a pale yellow color. After extractive removal of unconverted reactants, a virtually colorless product with 99% purity was obtained with 94% yield.

### Example 6

Preparation of N,N-dimethyl-4-methoxyphenylacetamide by Thermal Condensation (Comparative Example)

A melt of the 4-methoxyphenylacetic acid N,N-dimethylammonium salt was prepared by the method described in the preceding example. 400 g of toluene were added to this melt (400 g), and the mixture was heated to 150° C. With the aid of a water separator, the water of reaction formed in the amidation was separated out. After boiling under reflux for 48 hours, toluene was distilled off and the conversion was determined. Based on the acid used, a conversion of less than 2% was found. In addition, there was significant darkening of the reaction mixture.

### Example 7

Preparation of N,N-dimethyl-4-methoxyphenylacetamide by Thermal Condensation in the Presence of Iron Filings (Comparative Example)

The experiment according to example 6 was repeated, except that 1 g of iron filings were added to the reaction mixture. Again, the mixture was boiled at the boiling point of the toluene on a water separator for 48 hours. Based on the acid used, a conversion of less than 2% was again found. After the iron filings had been filtered off and the toluene had been removed by distillation, the reaction mixture contained 85 ppm of dissolved iron and had a black-brown color.

### Example 8

Preparation of N,N-dimethyl-4-methoxyphenylacetamide in a Batchwise Single-Mode Laboratory Microwave Apparatus (Comparative Example)

A melt of the 4-methoxyphenylacetic acid N,N-dimethylammonium salt was prepared by the method described in the preceding example. 2 ml of this melt were sealed pressure-tight in a pressure-tight vial and introduced into the microwave cavity of a “Biotage Initiator™” laboratory microwave unit. The reaction mixture was subsequently heated to 235° C. within one minute by applying 300 watts of microwave power, in the course of which a pressure of about 20 bar developed. After the end of the heating time, the sample was irradiated with regulated power for a further 300 seconds (5 minutes). In the course of this, the power was adjusted such that the temperature of the reaction mixture remained constant at 235° C. Based on the acid used, a conversion of 11% was found in the crude product.

1. A continuous process for preparing an amide comprising the steps of reacting at least one carboxylic acid of the formula I
R³—COON (I)
wherein R³ is hydrogen or a substituted or unsubstituted alkyl group having 1 to 4 carbon atoms
with at least one amine of the formula II
HNR¹R² (II)
wherein R¹ and R² are each independently hydrogen or a hydrocarbon radical having 1 to 100 carbon atoms
forming an ammonium salt and subsequently converting this ammonium salt to the fatty acid amide under microwave irradiation in a reaction tube whose longitudinal axis is in the direction of propagation of the microwaves from a monomode microwave applicator.

2. A process as claimed in claim 1, wherein the salt is irradiated with microwaves in a substantially microwave-transparent reaction tube within a hollow conductor connected via waveguides to a microwave generator.

3. A process as claimed in claim 1, wherein the microwave applicator is configured as a cavity resonator.

4. A process as claimed in claim 1, wherein the microwave applicator is configured as a cavity resonator of the reflection type.

5. A process as claimed in claim 1, wherein the reaction tube is aligned axially with a central axis of symmetry of the hollow conductor.

6. A process as claimed in claim 1, wherein the salt is irradiated in a cavity resonator with a coaxial transition of the microwaves.

7. A process as claimed in claim 1, wherein the cavity resonator is operated in E₀₁ₙ mode where n is an integer from 1 to 200.

8. A process as claimed in claim 1, wherein R³ is an alkyl group which has 1 to 4 carbon atoms and least one substituent selected from the group consisting of C₁-C₅-alkoxy, ester, amide, carboxyl, cyano, nitrile, nitro and C₅-C₂₀-aryl groups.

9. A process as claimed in claim 8, where the C₅-C₂₀-aryl groups have substituents selected from the group consisting of halogen atoms, C₁-C₂₀-alkyl, C₂-C₂₀-alkenyl, C₁-C₅-alkoxy, ester, amide, carboxyl, cyano, nitrile and nitro groups.

10. A process as claimed in claim 1, wherein R¹ and R² are each independently a hydrocarbon radical having 1 to 100 carbon atoms.

11. A process as claimed in claim 1, wherein R¹ is a hydrocarbon radical having 1 to 100 carbon atoms and R² is hydrogen.

12. A process as claimed in claim 1, wherein R¹ or R² or both have substituents selected from the group consisting of hydroxyl, C₁-C₅-alkoxy, cyano, nitrile, nitro and C₅-C₂₀-aryl groups.

13. A process as claimed in claim 1, wherein R¹ or R² or both have C₅-C₂₀-aryl groups, and the C₅-C₂₀-aryl groups have at least one substituent selected from the group consisting of halogen atoms, C₁-C₂₀-alkyl, C₂-C₂₀-alkenyl, hydroxyl, C₁-C₅-alkoxy, alkoxyalkyl, ester, amide, cyano, nitrile and nitro-substituted phenyl radicals.

14. A process as claimed in claim 1, wherein R¹ and R² together with the nitrogen atom to which they are bonded form a ring.

15. A process as claimed in claim 1, wherein R¹ and R² are each independently a radical of the formula III
—(R⁴—O)ₙ—R⁵ (III)
wherein
R⁴ is an alkylene group having 2 to 6 carbon atoms,
R⁵ is hydrogen or a hydrocarbon radical having 1 to 24 carbon atoms or a group of the formula —NR¹⁰R¹¹,
n is an integer from 2 to 50 and
R¹⁰, R¹¹ are each independently hydrogen, an aliphatic radical having 1 to 24 carbon atoms, an aryl group or heteroaryl group having 5 to 12 ring members, a poly(oxyalkylene) group having 1 to 50 poly(oxyalkylene) units, where the poly(oxyalkylene) units derive from alkylene oxide units having 2 to 6 carbon atoms, or R¹⁰ and R¹¹ together with the nitrogen atom to which they are bonded form a ring having 4, 5, 6 or more ring members.

16. A process as claimed in claim 1, wherein R¹ and R² are each independently a radical of the formula IV
—[R⁶—N(R⁷)]ₘ—(R⁷) (IV)
wherein
R⁶ is an alkylene group having 2 to 6 carbon atoms or mixtures thereof,
each R⁷ is independently hydrogen, an alkyl or hydroxyalkyl radical having up to 24 carbon atoms, a polyoxyalkylene radical —(R⁴—O)ₚ—R⁵, or a polyiminoalkylene radical —[R⁶—N(R⁷)]_{q}-(R⁷), where R⁴, R⁵, R⁶ and R⁷ are each as defined above and q and p are each independently 1 to 50, and
m is from 1 to 20.

17. A process as claimed in claim 1, wherein the microwave irradiation is performed at temperatures between 150 and 500° C.

18. A process as claimed in claim 1, wherein the microwave irradiation is performed at pressures above atmospheric pressure.

19. A process as claimed in claim 1, wherein R¹ or R² or both substituents are independently an aliphatic radical having 1 to 24 carbon atoms.

20. A process as claimed in claim 15, wherein R¹⁰ and R¹¹ are each independently an aliphatic radical having 2 to 18 carbon atoms.

21. A process as claimed in claim 16, wherein m is from 2 to 10.
